(19) United States
(12) Patent Application Publication
(10) Pub. No.: US 2013/0131613 A1
(43) Pub. Date: May 23, 2013

(54) Disposable Eye Mask Slip

(71) Applicant: Laura Susan Elkins, Frisco, TX (US)

(72) Inventor: Laura Susan Elkins, Frisco, TX (US)

(21) Appl. No.: 13/471,289

(22) Filed: May 14, 2012

Related U.S. Application Data
(60) Provisional application No. 61/527,261, filed on Aug. 25, 2011.

(51) Int. Cl. A61M 35/00

(52) U.S. Cl. CPC A61M 35/00
USPC 604/303

(57) ABSTRACT

An eye mask slip device to dispense the contents of a gel pack to a user may include a gel pack pouch to house the gel pack and a pair of ear arms to connect to the gel pack pouch to extend over the ear of the user. The gel pack pouch may include a back wall being permeable to dispense the contents of the gel pack. The gel pack pouch may include a front wall and a sidewall which may connect the front wall and back wall and extend around the periphery of the front wall and the wall. The sidewall may include a slit to allow the gel pack to be inserted and removed from the gel pack pouch. The gel pack pouch may include a fastener to close the slit.

### PRIORITY

The present invention claims priority under 35 USC section 119 and based upon the provisional application which was filed on Aug. 25, 2011 with a Ser. No. of 61/527,261.

### FIELD OF THE INVENTION

The present invention relates to an eye mask and more particularly to an eye mask having a gel pack pouch to dispense treatment.

### BACKGROUND

The following patents and patent applications are incorporated by reference in their entirety.

U.S. Pat. No. 6,823,868 discloses a travel mask that fits over the nose and mouth area of a wearer's face for filtration of air, particularly recycled air in the travel compartment of a common carrier, the travel mask having a face shield that preferably includes an aroma patch that emits a pleasant aroma and a retainer such as a head band for retaining the face shield against a user's head. The retainer has slidable ear plugs for blocking noise. The travel mask also has a foldable eye flap or flaps for blocking light when the flap or flaps are extended, the flap or flaps preferably having thermal gel packs for cooling or warming the eye lid area of the wearer during use.

U.S. Pat. No. 5,553,321 discloses a sun visor and eyeglasses case adapted to be attached to the temples of a pair of eyeglasses. In particular the sun visor is constructed of two plies of pliable material in an overlying relationship. Unattached ends of the sun visor in combination with an opening in the lower ply enables temple arms to be inserted between layers conveniently, thus forming a stable connection. The opening in the lower ply of material allows the lenses of eyeglasses to be inserted directly into the protective space between the plies without separating the eyeglass arms from sun visor. The sun visor may also serve as an eyeglasses float.

U.S. Pat. No. 5,102,216 discloses a stretch fabric being double rolled and then sewn to tubular straps at each end to form an eyeglasses retainer, case combination. The tubular straps are attached to the stems of eyeglasses and are held in place on the stems by elastic bands within the ends of the straps. The assembly is then worn around the head or neck as a retainer preventing the eyeglasses from sliding forward or falling off in sports and such. When the eyeglasses are not worn they can be folded and placed within the cavity formed by unrolling the double roll of the case.

U.S. Pat. No. 4,527,565 discloses a cold facial compress in the form of a mask having two sheets of flexible material bonded together along common edges and contains a gel in the space formed therebetween. The compress including the gel remains pliable at temperatures below that at which water freezes and may be maintained in the freezer section of a refrigerator and thereafter applied to the face of the user. The mask includes a notch adapted to be disposed beneath and about the nose of the user, an elongated opening adopted to be positioned about the lips of the user and a sharp notch disposed beneath the nose receiving notch and the slot. The mask is symmetrical about a line extending through the central portion of the notches and the slot and has a shape such as to be used by persons having faces of various sizes and shapes.

US patent application 20050229281 discloses a device to permit a eye mask, eyes cover, face shield or mask, sleep shade or sleep mask, light shade, blindfold, eye patch for a single eye and/or other such facial cover, to hold a single or a grouping of hygienic plastic, paper or other hygienic material in a removeable fashion so that easy cleaning can be effected after use and/or the hygienic article can be discarded so that the post-use impure material does not contact again the same or a different user's face. The device of this invention may also be impregnated with a scent and/or a cream, gel or other element to impart a substance or treatment onto eyes and/or face over a prolonged period.

### SUMMARY

An eye mask slip device to dispense the contents of a gel pack to a user may include a gel pack pouch to house the gel pack and a pair of ear arms to connect to the gel pack pouch to extend over the ear of the user.

The gel pack pouch may include a back wall being permeable to dispense the contents of the gel pack.

The gel pack pouch may include a front wall and a sidewall which may connect the front wall and back wall and extend around the periphery of the front wall and the wall.

The sidewall may include a slit to allow the gel pack to be inserted and removed from the gel pack pouch.

The gel pack pouch may include a fastener to close the slit.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a perspective view of the gel mask slip device of the present invention;

FIG. 2 illustrates a front view of the gel pack pouch of the gel mask slip device of the present invention;

FIG. 3 illustrates a back view of the gel pack pouch of the gel mask slip device of the present invention;

FIG. 4 illustrates a top view of the gel pack pouch of the gel mask slip device of the present invention;

FIG. 5 illustrates a bottom view of the gel mask slip device of the present invention;

FIG. 6 illustrates a side view of the gel mask slip device of the present invention;

FIG. 7 illustrates a another side view of the gel mask slip device of the present invention;

FIG. 8 illustrates a back perspective view of another gel mask slip device of the present invention;

FIG. 9 illustrates another gel mask slip device of the present invention.

### DETAILED DESCRIPTION

As mouth and nose masks are necessary to contain germs and bacteria from cross contamination, eye mask slips will provide sanitary use of eye masks among a variety of people.

The eye mask slip of the present invention may be disposable and a low cost per unit to encourage proper use among successive users.

Eye mask slip material may vary in thickness and layers dependent on the appropriate level of sanitation required by the user. The eye mask slip may be include materials that make up the face mask worn by a Dentist or nurse which may include a filter which may include multi-layers of web material.

Eye mask slip material may be transparent and may vary in and the degree of transparency from highly transparent (see-through) to light-blocking to accommodate the various environments for which they could be used. The user may be a dental patient or a spa client. A dental patient may desire a semi transparent mask to maintain awareness of the procedure, while a salon or spa client may desire total light-blocking to maximize restfulness.

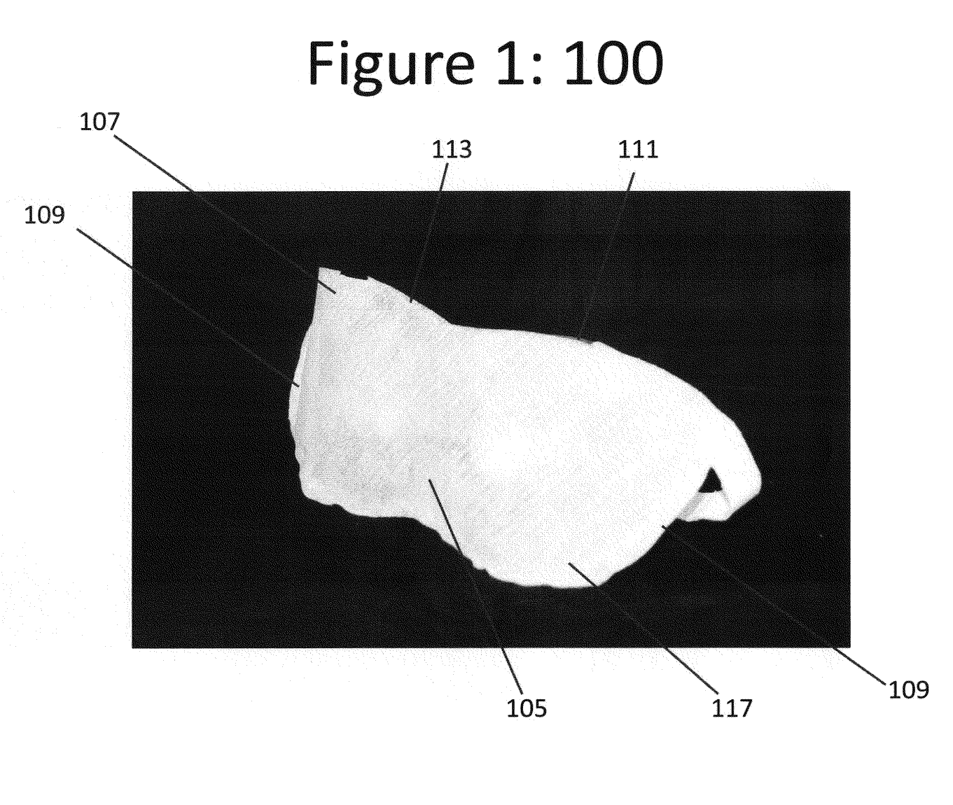

The eye mask slip may include a gel pack pouch 101 which may extend over a portion of the face which may correspond to the portion of the face which may be covered by glasses and may extend from approximately the nose of the user to the forehead of the user and may extend from approximately the side of the head of the user to the opposing side of the head of the user. The gel pack pouch 101 as shown in FIG. 1 may be hollow to form a cavity 117 in order to accommodate a gel pack 105 and may include a front wall 107 which may be connected to a side wall 109 which may extend around the periphery of the front wall 107 and the side wall 109 may be connected to a back wall 111 which may be connected to the sidewall 109 at the periphery of the back wall 111. The gel pouch 101 may include a gel pack slit 113 which may extend through the top surface of the sidewall 109 to allow the gel pack 105 to be inserted into the gel pack pouch 101 and removed from the gel pack pouch 101 when the life of the gel pack 105 has substantially ended. The gel pack pouch 101 may have a substantial rectangular cross-section.

The gel pack slit 113 may be closed by a fastener 115 which may include a button, a zipper, Velcro (hooks and loops) or other types of fasteners. The gel pack pouch 101 may be formed from flexible material such as leather, plastic and the gel pack pouch 101 may be formed from permeable material in order to allow the contents of the gel pack 105 to diffuse through the gel pack pouch 101 to the skin of the user. The gel pack 105 may be adapted to be warmed in order to provide heat for the user, may be adapted to be cooled in order to provide cooling for the user and may be adapted to include chemical agents to be applied to the skin of the user.

The eye mask slip 100 may include a pair of opposing ear arms 121 (or temple arms) which may be connected to the gel pack pouch 101 at the upper left and right hand side corner and may extend over the ear of the user. The opposing ear arms 101 may include an angled portion 123 which extend over the ear of the user. The ear arms 101 may be formed from rigid material such as metal, plastic, wood or other appropriate material or may be formed from flexible material or expendable material such as a fabric, stretchable rubber or other appropriate material and which may extend over the head of the user like a typical face mask worn by a dentist, or a fastener such as full head Velcro strap, or other fastening device to be tied together.

The strap may be a single strap and maybe formed from elastic material in order to adjust to different size heads.

The gel pack pouch 101 may include a fastener 115 or a closure tab on the top of the eye mask slip to ensure stability of the mask within if the wearer is horizontal or tilted towards the floor.

Mask slips to be made in various sizes to accommodate different types of use and different sizes of the soothing masks placed inside them.

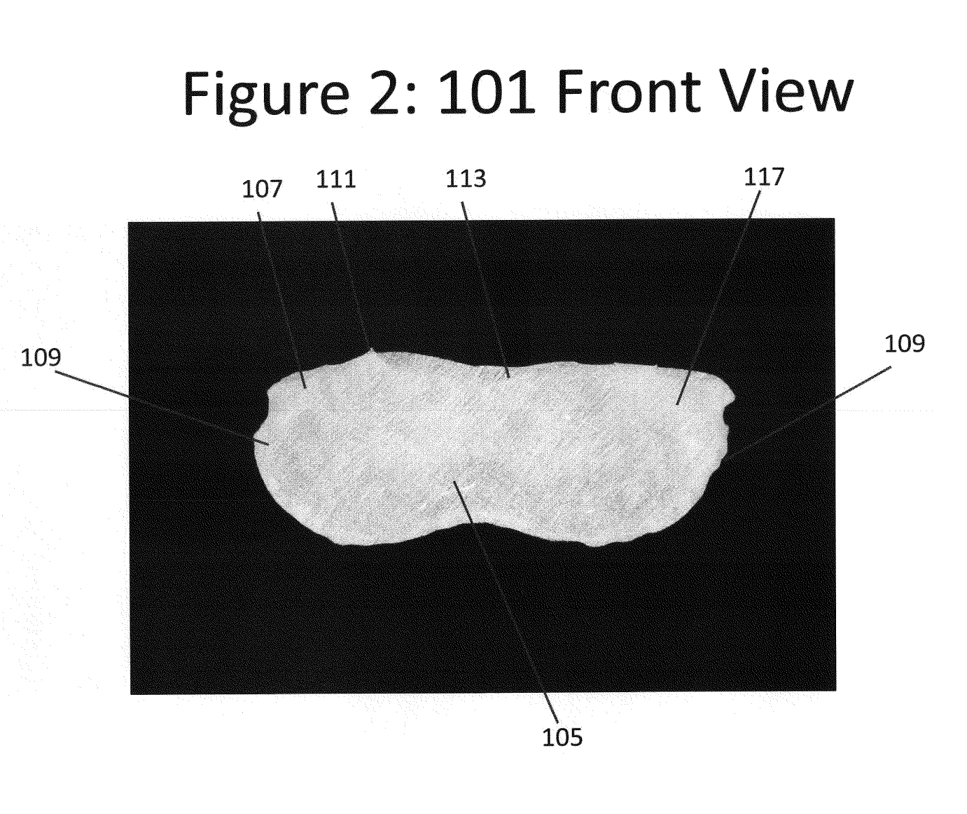

FIG. 2 illustrates the gel pack pouch 101 which may be hollow to form a cavity 117 in order to accommodate a gel pack 105 (not shown) and may include a front wall 107 which may be connected to a side wall 109 which may extend around the periphery of the front wall 107 and the side wall 109 may be connected to a back wall 111 which may be connected to the sidewall 109 at the periphery of the back wall 111. The gel pouch 101 may include a gel pack slit 113 which may extend through the top surface of the sidewall 109 to allow the gel pack 105 to be inserted into the gel pack pouch 101 and removed from the gel pack pouch 101 when the life of the gel pack 105 has substantially ended.

The gel pack pouch 101 may be formed from flexible material such as leather, plastic and the gel pack pouch 101 may be formed from permeable material in order to allow the gel pack 105 to diffuse through the gel pack pouch 101 to the skin of the user. The gel pack 105 may be adapted to be warmed in order to provide heat for the user, may be adapted to be cooled in order to provide cooling for the user and may be adapted to include chemical agents to be applied to the skin of the user.

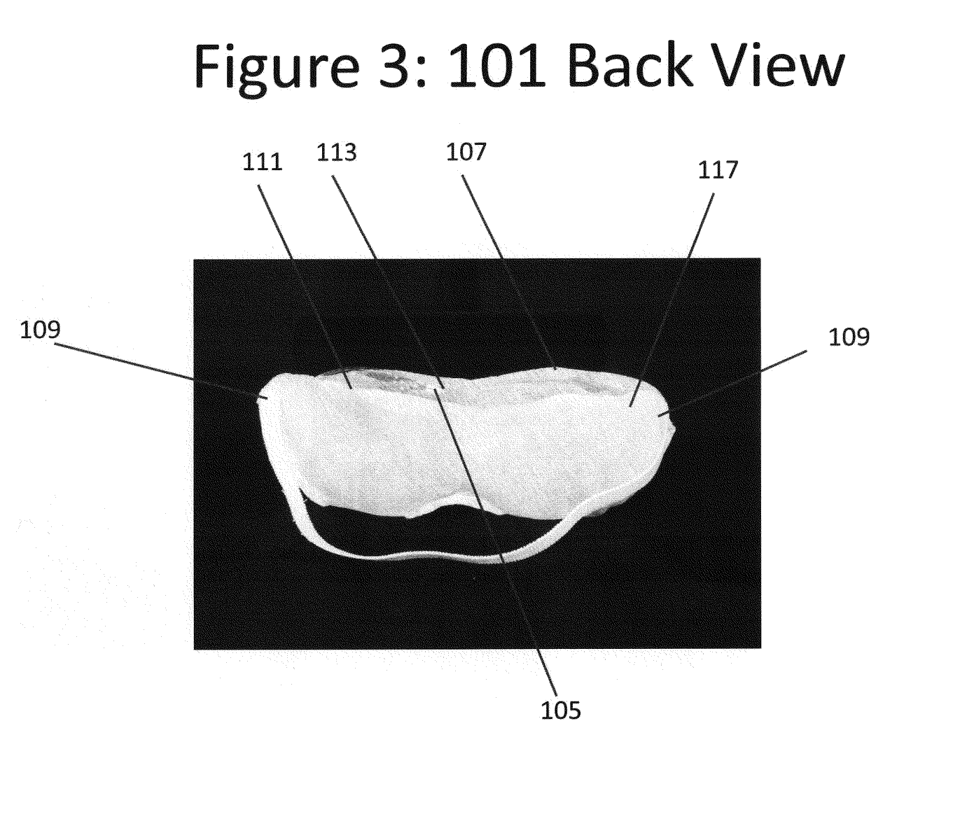

FIG. 3 illustrates a back view and illustrates the gel pack pouch 101 which may be hollow to form a cavity 117 in order to accommodate a gel pack 105 (not shown) and may include a front wall 107 which may be connected to a side wall 109 which may extend around the periphery of the front wall 107 and the side wall 109 may be connected to a back wall 111 which may be connected to the sidewall 109 at the periphery of the back wall 111. The gel pouch 101 may include a gel pack slit 113 which may extend through the top surface of the sidewall 109 to allow the gel pack 105 to be inserted into the gel pack pouch 101 and removed from the gel pack pouch 101 when the life of the gel pack 105 has substantially ended.

The gel pack pouch 101 may be formed from flexible material such as leather, plastic and the gel pack pouch 101 may be formed from permeable material in order to allow the gel pack 105 to diffuse through the gel pack pouch 101 to the skin of the user. The gel pack 105 may be adapted to be warmed in order to provide heat for the user, may be adapted to be cooled in order to provide cooling for the user and may be adapted to include chemical agents to be applied to the skin of the user.

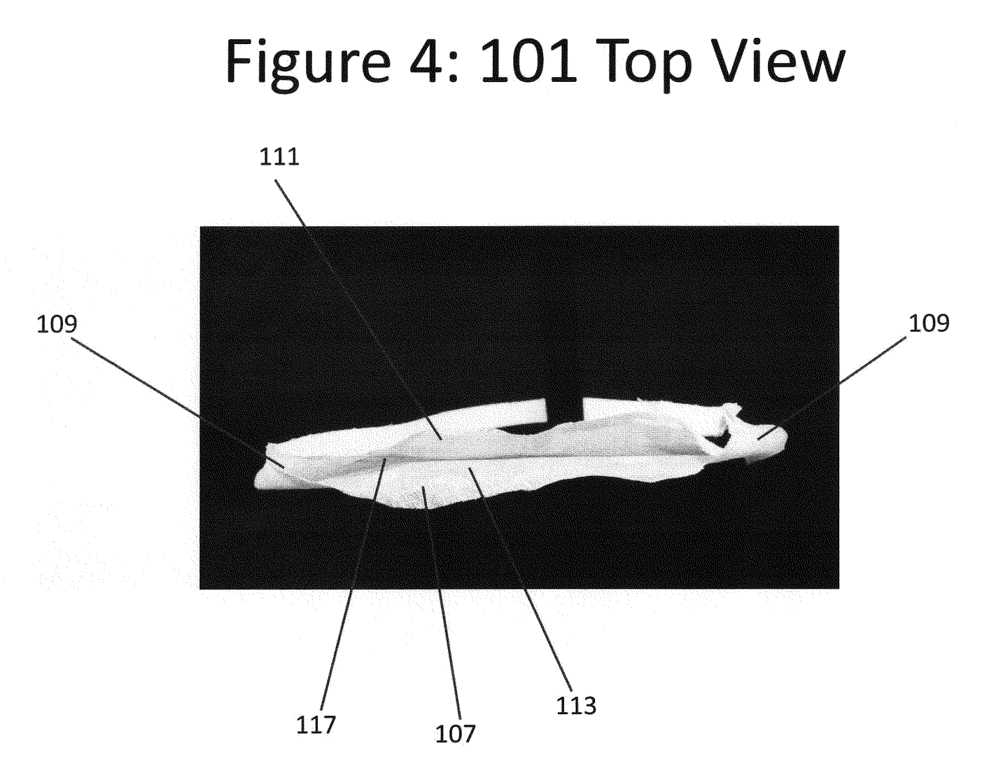

FIG. 4 illustrates a top view and illustrates the gel pack pouch 101 which may be hollow to form a cavity 117 in order to accommodate a gel pack 105 (not shown) and may include a front wall 107 which may be connected to a side wall 109 which may extend around the periphery of the front wall 107 and the side wall 109 may be connected to a back wall 111 which may be connected to the sidewall 109 at the periphery of the back wall 111. The gel pouch 101 may include a gel pack slit 113 which may extend through the top surface of the sidewall 109 to allow the gel pack 105 to be inserted into the gel pack pouch 101 and removed from the gel pack pouch 101 when the life of the gel pack 105 has substantially ended.

The gel pack pouch 101 may be formed from flexible material such as leather, plastic and the gel pack pouch 101 may be formed from permeable material in order to allow the gel pack 105 to diffuse through the gel pack pouch 101 to the skin of the user. The gel pack 105 may be adapted to be warmed in order to provide heat for the user, may be adapted to be cooled in order to provide cooling for the user and may be adapted to include chemical agents to be applied to the skin of the user.

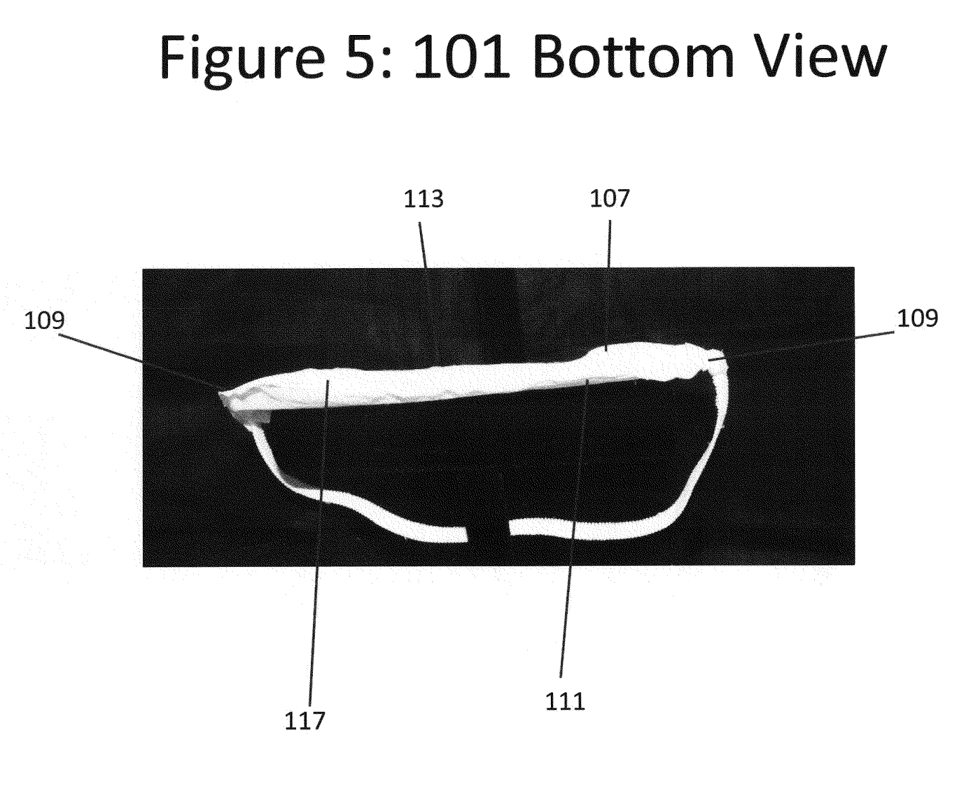

FIG. 5 illustrates a bottom view and illustrates the gel pack pouch 101 which may be hollow to form a cavity 117 in order to accommodate a gel pack 105 (not shown) and may include a front wall 107 which may be connected to a side wall 109 which may extend around the periphery of the front wall 107 and the side wall 109 may be connected to a back wall 111 which may be connected to the sidewall 109 at the periphery of the back wall 111. The gel pouch 101 may include a gel pack slit 113 which may extend through the top surface of the sidewall 109 to allow the gel pack 105 to be inserted into the gel pack pouch 101 and removed from the gel pack pouch 101 when the life of the gel pack 105 has substantially ended.

The gel pack pouch 101 may be formed from flexible material such as leather, plastic and the gel pack pouch 101 may be formed from permeable material in order to allow the gel pack 105 to diffuse through the gel pack pouch 101 to the skin of the user. The gel pack 105 may be adapted to be warmed in order to provide heat for the user, may be adapted to be cooled in order to provide cooling for the user and may be adapted to include chemical agents to be applied to the skin of the user.

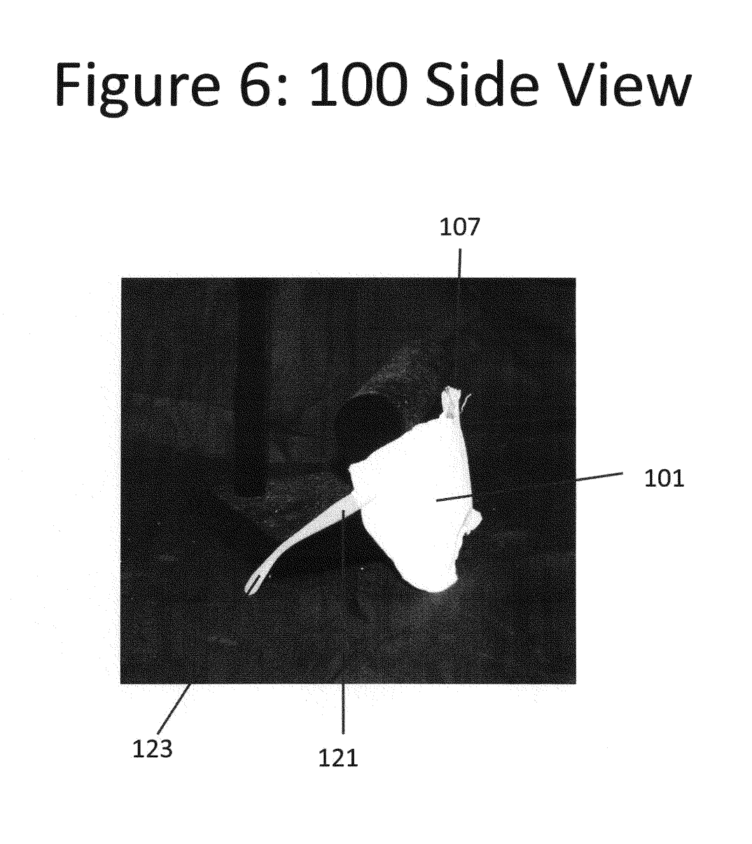

FIG. 6 illustrates a side view of the eye mask slip 100 and illustrates the gel pack pouch 101, the ear arms 121 and a angled portion 123

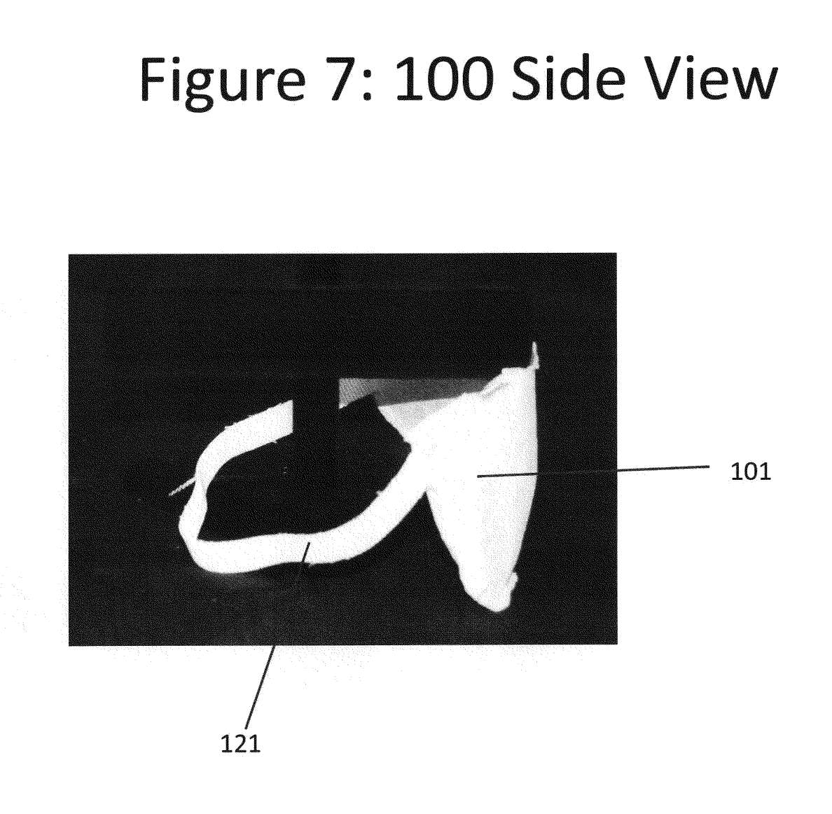

FIG. 7 illustrates a side view of the eye mask slip 100 and illustrates the gel pack pouch 101 and the ear arms 121 without the angled portion 123.

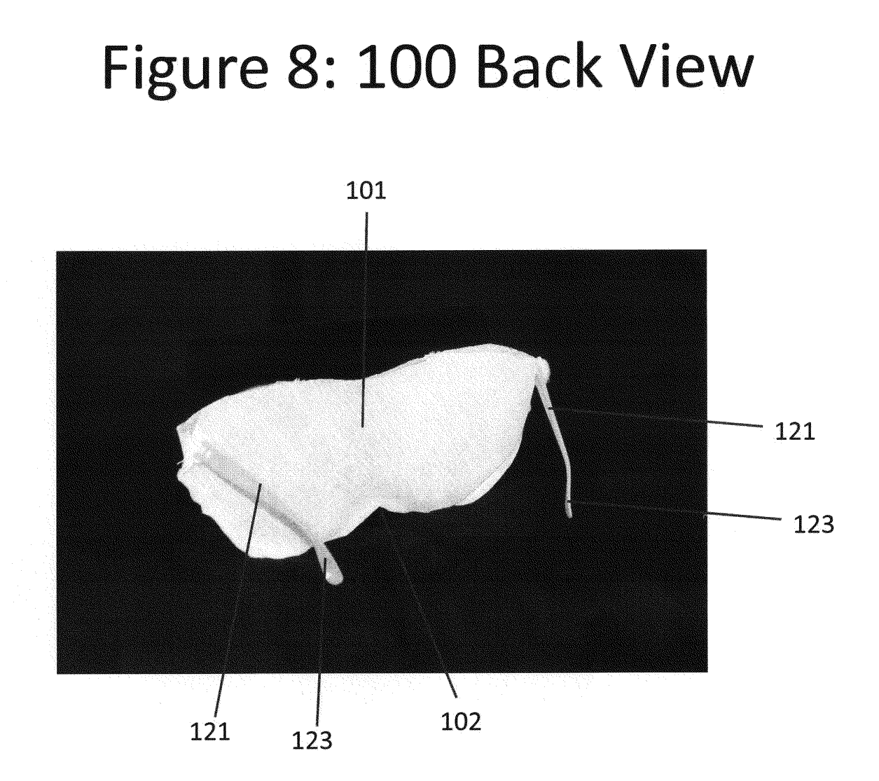

FIG. 8 illustrates a back perspective view of the eye mask slip 100 and illustrates the gel pack pouch 101, the eye arms 121 and the angled portion 123. The gel pack pouch includes a depression 102 to correspond to the nose of a user.

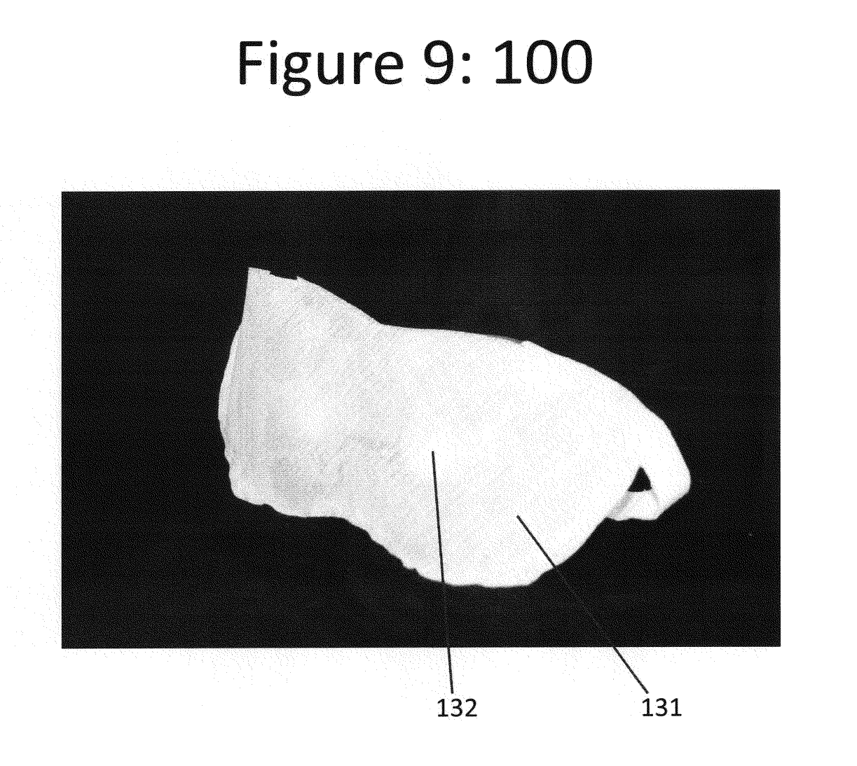

FIG. 9 illustrates a front perspective view of the eye mask slip 100 and illustrates the gel pack pouch 101 and the eye arms 121. The eye mask pouch 101 may include a first area 131 being substantially opaque to prevent light from the user and a second area 132 being substantially transparent to allow the user to see.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) An eye mask slip device to dispense the contents of a gel pack to a user, comprising:
a gel pack pouch to house the gel pack;
a pair of ear arms to connect to the gel pack pouch to extend over the ear of the user;
wherein the gel pack pouch includes a back wall being permeable to dispense the contents of the gel pack.

2) An eye mask slip device to dispense the contents of a gel pack to a user as in claim 1, wherein the gel pack pouch includes a front wall and a sidewall which may connect the front wall and back wall and extend around the periphery of the front wall and the wall.

3) An eye mask slip device to dispense the contents of a gel pack to a user as in claim 2, wherein the sidewall includes a slit to allow the gel pack to be inserted and removed from the gel pack pouch.

4) An eye mask slip device to dispense the contents of a gel pack to a user as in claim 3, wherein the gel pack pouch includes a fastener to close the slit.
